(19) United States
(12) Patent Application Publication
(10) Pub. No.: US 2011/0135661 A1
(43) Pub. Date: Jun. 9, 2011

(54) TREATMENT AND PREVENTION OF DRY AGE-RELATED MACULAR DEGENERATION BY ACTIVATING CD36

(75) Inventors: Huy Ong, Ville Mont-Royal (CA); Sylvain Chemtob, Cote St-Luc (CA); Florian Sennlaub, Paris (FR)

(73) Assignees: Universite De Montreal, Montreal, QC (CA); Centre Hospitalier Universitaire Sainte-Justine, Montreal, QC (CA); INSERM (Institut National de la Sante et de la Recherche Medicale), Paris (FR)

(21) Appl. No.: 12/735,808

(22) PCT Filed: Feb. 19, 2009
(86) PCT No.: PCT/CA2009/000200

§ 371 Date: Jan. 31, 2011

Related U.S. Application Data
(60) Provisional application No. 61/029,819, filed on Feb. 19, 2008.

(51) Int. Cl. A61K 39/395; A61K 38/08; C12Q 1/02; A61P 27/02

(52) U.S. Cl. 424/172.1; 514/20.8; 514/11.3; 435/29

(57) ABSTRACT

The present invention relates to methods and compositions for the prevention and/or treatment of dry age-related macular degeneration by administering a CD 36-activator compound to a subject in need thereof; wherein the CD36 activator compound is the CD36 antibody FA6-152, or the growth hormone (GH)-releasing peptide family-derived compound EP80317 or the compound of Formula 1 [A-(Xaa)ₐ-N(R^{A})—N(R^{B})—C(O)-(Xaa′)_{b}-B]; and wherein the activator compound acts to maintain a healthy choroid and retinal oxygenation by enhanced COX2 expression.

### FIELD OF THE INVENTION

The present invention concerns treatment and prevention of dry age-related macular degeneration by activating CD36.

### BACKGROUND OF THE INVENTION

Age-related macular degeneration (AMD) is the leading cause of vision loss among older adults in industrialized countries (1). The prevalence of AMD which is 0.05% before 50 years old, rises to 11.8% after 80 years of age and is expected to double in the coming decades because of the projected increase in aging population (2,3).

The causes of AMD are poorly understood, but it is agreed that the progressive decline of vision in AMD results from the dysfunction of the central retina principally its underlying elements, the retinal pigment epithelium (RPE), the Bruch membrane (BM), the choriocapillaris and degeneration of the photoreceptors (4). Other than age, few predisposing factors have been clearly identified; these include light, cigarette smoking, possibly hypertension and atherosclerosis (5). In this context, despite their specific characteristics an analogy between deposits found in AMD and atherosclerosis has been proposed (6).

Early AMD is characterized by drusen (focal deposits in BM) and basal deposits (diffuse sub-RPE debris in BM) and changes in RPE pigmentation. Two clinical forms of late AMD have been identified: the “dry” form, characterized by circumscribed atrophy of RPE and thinning and obliteration of the choriocapillary layer and the “wet” form defined by choroidal neovascularization, and (geographic atrophy) (4, 7). Although most of the cases of legal blindness in AMD are consequence of choroidal neovascularization (the wet form), the vast majority of patients first develop severe visual impairment secondary to geographic atrophy seen in the dry form (8).

Current research and emerging therapies (anti-vascular endothelial growth factor [VEGF] treatments) mainly focus on the neovascular aspect of wet AMD and little treatment is available to patients with the atrophic, dry form. A major National Eye Institute study (AREDS) has produced some evidence that certain nutrients such as beta carotene (vitamin A) and vitamins C and E may help to prevent or slow progression of dry AMD, although the protective effect of the antioxidant cocktail of zinc, vitamins C, E and β-carotene was modest and evident only in a subgroup of patients. Eye doctors also recommend that dry AMD patients wear sunglasses with UV protection against potentially harmful effects of the sun. However, no FDA-approved treatments are available for dry AMD, therefore there is still a need to develop strategies to treat dry macular degeneration.

The basic mechanisms underlying AMD, and particular geographic atrophy and choroidal involution, remain elusive. Physiologically, the RPE cells transfer oxygen and nutrients from the choroidal circulation to the outer retina (external hemato-retinal barrier). Although the loss of vision is caused by photoreceptor degeneration, it is thought that atrophic AMD is trigged by an insult to the RPE. RPE abnormalities may be caused by oxidative stress, local inflammation, formation of drusen or, potentially, by excessive accumulation of the autofluorescent pigment lipofuscin. RPE engulf, degrade, and recycle used photoreceptor outer segment (OS), and clear the debris to the choroidal circulation. Phagocytosis of spent OS is critical for the long-term maintenance of the retina (9,10) and is dependent on a tyrosine kinase receptor (i.e., for c-mer proto-oncogene tyrosine kinase [MERTK]) (11, 12) and integrins (13). CD36 is a type B scavenger receptor (14) that is expressed in RPE cells (15), among others. CD36 is involved in phagocytosis (16) particularly of oxidized lipids (17). Phagocytosis in turn “induces” a number of genes expressed in RPE (18) such as the proangiogenic cyclooxygenase 2 COX2 (19) (also known as prostaglandin-endoperoxide synthase 2 [PTGS2]), which controls VEGF expression in various cells (20). In addition, the multiple-ligand receptor CD36 is the main antiangiogenic receptor of thrombospondin-1 (TSP-1) (21).

### SUMMARY OF THE INVENTION

The present inventors have found a novel treatment for dry AMD. Pharmacological activation of CD36 in the RPE of patients with dry AMD can be used to prevent photoreceptor cell death and to maintain a healthy choroid and retinal oxygenation by enhanced COX2 expression.

Deficiency of CD36, which participates in OS phagocytosis by the RPE in vitro, leads to significant progressive age-related photoreceptor degeneration evaluated histologically at different ages in two rodent models of CD36 invalidation in vivo (Spontaneous hypertensive rats (SHR) and CD36^{-/-} mice). Furthermore, these animals developed significant age related choroidal involution reflected in a 100%-300% increase in the avascular area of the choriocapillaries measured on vascular corrosion casts of aged animals. Proangiogenic cyclooxygenase-2 (COX2) expression in RPE is stimulated by CD36 activating antibody and that CD36-deficient RPE cells from SHR rats fail to induce COX2 and subsequent VEGF expression upon OS or antibody stimulation in vitro. CD36^{-/-} mice express reduced levels of COX2 and VEGF in vivo, and COX2^{-/-} mice develop progressive choroidal degeneration similar to what is seen in CD36 deficiency.

According to one an aspect of the present invention there is provided a method for preventing or treating dry age-related macular degeneration in a subject which comprises administering a therapeutically effective amount of a CD36 activator compound to a subject.

From a yet further aspect, there is a provided a method to prevent or treat dry AMD in a human or a non-human mammal.

It is understood that by a non-human mammal, it is meant a mammal such cats, dogs, swine, cattle, sheep, goats, horses, rabbits, rats, mice and the like.

From another aspect, there is a method to prevent and treat dry AMD with a CD36 activator antibody, FA6-152.

CD36 activator compounds are known in the art and it is understood that they would be suitable for the purpose of the present invention. Such CD36 activator compounds can be, for example, compounds of Formula I defined as:

A-(Xaa)ₐ-N(R^{A})—N(R^{B})—C(O)-(Xaa′)_{b}-B I

wherein

a is an integer from 0 to 5;

b is an integer from 0 to 5;

Xaa and Xaa′ are each any D or L amino acid residue or a D,L amino acid residue mixture;

A is

1) H,

2) C₁-C₆ alkyl,

3) C₂-C₆ alkenyl,

4) C₂-C₄ alkynyl,

5) C₃-C₇ cycloalkyl,

6) haloalkyl,

7) heteroalkyl,

8) aryl,

9) heteroaryl,

10) heteroalkyl,

11) heterocyclyl,

12) heterobicyclyl,

13) C(O)R³,

14) SO₂R³,

15) C(O)OR³, or

16) C(O)NR⁴R⁵,

wherein the alkyl, the alkenyl, the alkynyl and the cycloalkyl are optionally substituted with one or more R¹ substituents; and wherein the aryl, the heteroaryl, the heterocyclyl and the heterobicyclyl are optionally substituted with one or more R² substituents;

B is

1) OH,

2) OR³, or

3) NR⁴R⁵;

R^{A} and R^{B} are independently chosen from

1) H,

2) C₁-C₆ alkyl,

3) C₂-C₆ alkenyl,

4) C₂-C₆ alkynyl,

5) C₃-C₇ cycloalkyl,

6) C₅-C₇ cycloalkenyl,

7) haloalkyl,

8) heteroalkyl,

9) aryl,

10) heteroaryl,

11) heterobicyclyl, or

12) heterocyclyl,

wherein the alkyl, alkenyl, alkynyl and the cycloalkyl and cycloalkenyl are optionally substituted with one or more R¹ substituents; and wherein the aryl, the heteroaryl, the heterocyclyl and the heterobicyclyl are optionally substituted with one or more R² substituents,

or alternatively, R^{A} and R^{B} together with the nitrogen to which each is bonded form a heterocyclic or a heterobicyclic ring;

R¹ is

1) halogen,

2) NO₂,

3) CN,

4) haloalkyl,

5) C₃-C₇ cycloalkyl,

6) aryl,

7) heteroaryl,

8) heterocyclyl,

9) heterobicyclyl,

10) OR⁶,

11) S(O)₂R³,

12) NR⁴R⁵,

13) NR⁴S(O)₂R³,

14) COR^{E},

15) C(O)OR⁶,

16) CONR⁴R⁵,

17) S(O)₂NR⁴R⁵,

18) OC(O)R⁶,

19) SC(O)R³,

20) NR⁶C(O)NR⁴R⁵,

21) heteroalkyl,

22) NR⁶C(NR⁶)NR⁴R⁵, or

23) C(NR⁶)NR⁴R⁵;

wherein the aryl, heteroaryl, heterocyclyl, and heterobicyclyl are optionally substituted with one or more R² substituents;

R² is

1) halogen,

2) NO₂,

3) CN,

4) C₁-C₆ alkyl,

5) C₂-C₆ alkenyl,

6) C₂-C₄ alkynyl,

7) C₃-C₇ cycloalkyl,

8) haloalkyl,

9) OR⁶,

10) NR⁴R⁵,

11) SR⁶,

12) COR⁶,

13) C(O)OR⁶,

14) S(O)₂R³,

15) CONR⁴R⁵,

16) S(O)₂NR⁴R⁵,

17) aryl,

18) heteroaryl,

19) heterocyclyl,

20) heterobicyclyl,

21) heteroalkyl,

22) NR⁶C(NR⁶)NR⁴R⁵, or

23) C(NR⁶)NR⁴R⁵,

wherein the aryl, the heteroaryl, the heterocyclyl, and the heterobicyclyl are optionally substituted with one or more R⁷ substituents;

R³ is

1) C₁-C₆ alkyl,

2) C₂-C₆ alkenyl,

3) C₂-C₄ alkynyl,

4) C₃-C₇ cycloalkyl,

5) haloalkyl,

6) aryl,

7) heteroaryl,

8) heterocyclyl, or

9) heterobicyclyl,

wherein the alkyl, the alkenyl, the alkynyl and the cycloalkyl are optionally substituted with one or more R¹ substituents; and wherein the aryl, the heteroaryl, the heterocyclyl and the heterobicyclyl are optionally substituted with one or more R² substituents;

R⁴ and R⁵ are independently chosen from

1) H,

2) C₁-C₆ alkyl,

3) C₂-C₆ alkenyl,

4) C₂-C₆ alkynyl,

5) aryl,

6) heteroaryl, or

7) heterocyclyl,

or R⁴ and R⁵ together with the nitrogen to which they are bonded form a heterocyclic ring;

R⁶ is

1) H,

2) C₁-C₆ alkyl,

3) C₂-C₆ alkenyl,

4) C₂-C₆ alkynyl,

5) aryl,

6) heteroaryl, or

7) heterocyclyl;

R⁷ is

1) halogen,

2) NO₂,

3) CN,

4) C₁-C₆ alkyl,

5) C₂-C₆ alkenyl,

6) C₂-C₄ alkynyl,

7) C₃-C₇ cycloalkyl,

8) haloalkyl,

9) OR⁶,

10) NR⁴R⁵,

11) SR⁶,

12) COR⁶,

13) C(O)OR⁶,

14) S(O)₂R³,

15) CONR⁴R⁵,

16) S(O)₂NR⁴R⁵,

17) heteroalkyl,

18) NR⁶C(NR⁶)NR⁴R⁵, or

19) C(NR⁶)NR⁴R⁵;

or a salt thereof, or a prodrug thereof;

wherein the following compounds are excluded:

### A is H, (Xaa)ₐ is His-D-Trp, R^{A} is H, R^{B} is CH₃, (Xaa′)_{b} is Trp-D-Phe-Lys and B is NH₂;

### A is H, (Xaa)ₐ is His-D-Trp-Ala-Trp, R^{A} is H, R^{B} is CH₂Ph, (Xaa′)_{b} is Lys and B is NH₂;

A is H, (Xaa)ₐ is (D/L)-His, R^{A} is H, R^{B} is CH₂-p-C₆H₄OH, (Xaa′)_{b} is Ala-Trp-D-Phe-Lys and B is NH₂;
A is H, (Xaa)ₐ is His-D-Trp-Ala, R^{A} is H, R^{B} is CH₂-p-C₆H₄OH, (Xaa′)_{b} is D-Phe-Lys and B is NH₂; and

### A is H, (Xaa)ₐ is His-D-Trp-Ala-D-Phe, R^{A} is H, R^{B} is (CH₂)₄NH_{2, b} is 0, and B is NH₂.

Such CD36 activator compounds can also be, for example, EP80317 derived from the growth hormone (GH)-releasing peptide family. It is understood that one skilled in the art would readily know if a compound is a CD36 activator and would therefore identify that compound as being suitable for the present invention.

Such compounds are preferably administered systemically (intraperitoneally or intravenously) or locally (intravitreally or intraocularly) in a submicromolar concentration in preferred dosage of about 300 μg/kg.

In another aspect, there is provided a composition for preventing or treating dry age-related macular degeneration comprising a CD36 activator compound in association with a pharmaceutically acceptable carrier.

As used herein, the term “comprising” is intended to mean that the list of elements following the word “comprising” are required or mandatory but that other elements are optional and may or may not be present.

As used herein, the term “pharmaceutically acceptable carrier”, diluent or excipient” is intended to mean, without limitation, any adjuvant, carrier, excipient, glidant, diluent preservative, dye/colorant, aerosol spray, flavor enhancer, surfactant, wetting agent, dispersing agent, suspending agent, stabilizer, isotonic agent, solvent, emulsifier, or encapsulating agent, such as liposome, cyclodextrins, encapsulating polymeric delivery systems or polyethylene glycol matrix, which is acceptable for use in the subject, preferably humans.

As used herein, the term “treating” or “treatment” is intended to mean treatment of a disease-state in which activation of CD36 is desired, as disclosed herein, in a subject, and includes, for example; i) preventing a disease or condition, in which activation of CD36 is desired, from occurring in a subject, in particular, when such mammal is predisposed to the disease or condition but has not yet been diagnosed as having it; ii) inhibiting a disease or condition associated with CD36 activity, i.e. arresting its development; or iii) relieving a disease or condition associated with CD36 activity, i.e. causing regression of the condition.

As used herein, the term “therapeutically effective amount” is intended to mean an amount of a CD36 activator which, when administered to a subject is sufficient to effect treatment for a disease-state in which activation of CD36 is desired. The of CD36 activator will vary depending on the compound, the condition and its severity, and the age of the subject to be treated, but can be determined routinely by one of ordinary skill in the art having regard to his own knowledge and to this disclosure.

As used herein, the term “amino acid” is intended to mean at least any of the following α-amino acids:

| Amino acid | Abbreviation |
|---|---|
| Alanine | Ala |
| Arginine | Arg |
| Aspartic acid | Asp |
| Asparagine | Asn |
| Cysteine | Cys |
| Glutamic acid | Glu |
| Glutamine | Gln |
| Glycine | Gly |
| Isoleucine | Ile |
| Histidine | His |
| Leucine | Leu |
| Lysine | Lys |
| Methionine | Met |
| Phenylalanine | Phe |
| Proline | Pro |
| Serine | Ser |
| Threonine | Thr |
| Tryptophan | Trp |
| Tyrosine | Tyr |
| Valine | Val |

The above list is not exclusive and it should be understood that other amino acids not listed above are included in the definition of amino acid, such as hydroxyproline, citruline, ornithine etc.

The natural amino acids, with the exception of glycine, contain a chiral carbon atom. Unless otherwise stated, the compounds of Formula I containing amino acids can be of either the L- or D-configuration, or can be mixtures of D- and L-isomers, including racemic mixtures. Additional non-natural amino acid residues which are contemplated include, but are not limited to, α-alkyl, α,α-dialkyl, α-aryl and α-heteroarylglycine analogs, aryl and heteroarylalanine analogs, β,β-dialkylcysteine analogs, β,β-dialkylserine analogs, branched leucine analogs, ornithine, cirtuline, sarcosine, allylglycine, aminobutyric acid, amino-iso-butyric acid, cyclohexylalanine, cyclohexylglycine (also named: 2-amino-2-cyclohexylacetic acid), norvaline, pipecolic acid, tert-butylglycine, and the like. Also included are β-amino acids such as beta-alanine, beta-homophenylalanine as well as longer chain amino acids such as gamma-aminobutyric acid.

As used herein, the term “residue” when referring to α-amino acids is intended to mean a radical derived from the corresponding α-amino acid by eliminating the hydroxyl of the carboxy group and one hydrogen of the α-amino group. For example, the terms Gln, Ala, Gly, Ile, Arg, Asp, Phe, Ser, Leu, Cys, Asn, and Tyr represent the residues of glutamine, alanine, glycine, isoleucine, arginine, aspartic acid, phenylalanine, serine, leucine, cysteine, asparagine, and tyrosine, respectively.

As used herein, the term “alkyl” is intended to include both branched and straight chain saturated aliphatic hydrocarbon groups having the specified number of carbon atoms, for example, C₁-C₆ as in C₁-C₆-alkyl is defined as including groups having 1, 2, 3, 4, 5 or 6 carbons in a linear or branched arrangement. Examples of alkyl as defined above include, but are not limited to, methyl, ethyl, n-propyl, i-propyl, n-butyl, t-butyl, i-butyl, pentyl and c-hexyl.

As used herein, the term, “alkenyl” is intended to mean unsaturated straight or branched chain hydrocarbon groups having the specified number of carbon atoms therein, and in which at least two of the carbon atoms are bonded to each other by a double bond, and having either E or Z regeochemistry and combinations thereof. For example, C₂-C₆ as in C₂-C₆ alkenyl is defined as including groups having 1, 2, 3, 4, 5, or 6 carbons in a linear or branched arrangement, at least two of the carbon atoms being bonded together by a double bond. Examples of C₂-C₆ alkenyl include ethenyl (vinyl), 1-propenyl, 2-propenyl, 1-butenyl and the like.

As used herein, the term “alkynyl” is intended to mean unsaturated, straight chain hydrocarbon groups having the specified number of carbon atoms therein and in which at least two carbon atoms are bonded together by a triple bond. For example C₂-C₄ as in C₂-C₄ alkynyl is defined as including groups having 2, 3, or 4 carbon atoms in a chain, at least two of the carbon atoms being bonded together by a triple bond.

As used herein, the term “cycloalkyl” is intended to mean a monocyclic saturated aliphatic hydrocarbon group having the specified number of carbon atoms therein, for example, C₃-C₇ as in C₃-C₇ cycloalkyl is defined as including groups having 3, 4, 5, 6 or 7 carbons in a monocyclic arrangement. Examples of C₃-C₇ cycloalkyl as defined above include, but are not limited to, cyclopropyl, cyclobutyl, cyclopentyl, cyclohexyl and cycloheptyl.

As used herein, the term “cycloalkenyl” is intended to mean unsaturated straight or branched chain hydrocarbon groups having the specified number of carbon atoms therein in a monocyclic arrangement, and in which at least two of the carbon atoms are bonded to each other by a double bond. For example, C₂-Cₛ as in C₂-C₈ cycloalkenyl is defined as having 2, 3, 4, 5, 6, 7 or 8 carbons in a monocyclic arrangement. Examples of cycloalkenyls as defined above include, but are not limited to cyclopentenyl, cyclohexenyl, cyclohexadienyl, cycloheptenyl, cyclooctenyl, and cyclooctadienyl.

As used herein, the term “halo” or “halogen” is intended to mean fluorine, chlorine, bromine and iodine.

As used herein, the term “haloalkyl” is intended to mean an alkyl, as defined above, in which each hydrogen atom may be successively replaced by a different halogen atom. Examples of haloalkyls include, but are not limited to, CH₂F, CHF₂ and CF₃.

As used herein, the term “heteroalkyl” is intended to mean a saturated linear or branched-chain monovalent hydrocarbon radical of one to six carbon atoms, wherein at least one of the carbon atoms is replaced with a heteroatom selected from N, O, or S, and wherein the radical may be a carbon radical or heteroatom radical (i.e., the heteroatom may appear in the middle or at the end of the radical). The heteroalkyl radical may be optionally substituted independently with one or more substituents described herein.

As used herein, the term “aryl”, either alone or in combination with another radical, means a carbocyclic aromatic monocyclic group containing 6 carbon atoms, which may be further fused to a second 5- or 6-membered carbocyclic group which may be aromatic, saturated or unsaturated. Aryl includes, but is not limited to, phenyl, indanyl, 1-naphthyl, 2-naphthyl and tetrahydronaphthyl. The fused aryls may be connected to another group either at a suitable position on the cycloalkyl ring or the aromatic ring. For example:

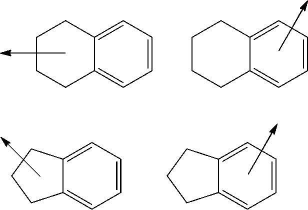

Arrowed lines drawn from the ring system indicate that the bond may be attached to any of the suitable ring atoms.

As used herein, the term “heteroaryl” is intended to mean a monocyclic or bicyclic ring system of up to ten atoms, wherein at least one ring is aromatic, and contains from 1 to 4 hetero atoms selected from the group consisting of O, N, and S. The heteroaryl substituent may be attached either via a ring carbon atom or one of the heteroatoms. Examples of heteroaryl groups include, but are not limited to thienyl, benzimidazolyl, benzo[b]thienyl, furyl, benzofuranyl, pyranyl, isobenzofuranyl, chromenyl, xanthenyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, napthyridinyl, quinoxalinyl, quinazolinyl, cinnolinyl, pteridinyl, isothiazolyl, isochromanyl, chromanyl, isoxazolyl, furazanyl, indolinyl, isoindolinyl, thiazolo[4,5-b]-pyridine, hydroxybenzotriazolyl, benzotriazoyl, triazoyl, and fluoroscein derivatives such as:

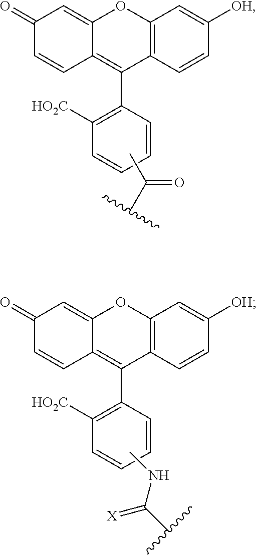

and rhodamine, dansyl and other fluorescent tags known to those skilled in the art.

As used herein, the term “heterocycle”, “heterocyclic” or “heterocyclyl” is intended to mean a non-aromatic ring system containing heteroatoms selected from the group consisting of O, N and S. Examples of aromatic heterocycles are described as heteroaromatic above. Examples of non-aromatic heterocycles include, but are not limited to azepinyl, azetidyl, aziridinyl, pyrrolidinyl, tetrahydrofuranyl, piperidyl, pyrrolinyl, piperazinyl, imidazolidinyl, morpholinyl, imidazolinyl, diazepinyl, pyrazolidinyl, pyrazolinyl, and biotinyl derivatives.

As used herein, the term “heterobicycle” either alone or in combination with another radical, is intended to mean a heterocycle as defined above fused to another cycle, be it a heterocycle, an aryl or any other cycle defined herein. Examples of such heterobicycles include, but are not limited to, pyrrolizidinyl, indolizidinyl, quinolizidinyl, coumarin, benzo[d][1,3]dioxole, 2,3-dihydrobenzo[b][1,4]dioxine and 3,4-dihydro-2H-benzo[b][1,4]dioepine.

As used herein, the term “optionally substituted with one or more substituents” or its equivalent term “optionally substituted with at least one substituent” is intended to mean that the subsequently described event of circumstances may or may not occur, and that the description includes instances where the event or circumstance occurs and instances in which it does not. The definition is intended to mean from zero to five substituents.

If the substituents themselves are incompatible with the synthetic methods of the present invention, the substituent may be protected with a suitable protecting group (PG) that is stable to the reaction conditions used in these methods. The protecting group may be removed at a suitable point in the reaction sequence of the method to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3^{rd} ed.), John Wiley & Sons, NY (1999), which is incorporated herein by reference in its entirety. Examples of protecting groups used throughout include, but are not limited to Alloc, Fmoc, Bn, Boc, CBz and COCF₃. In some instances, a substituent may be specifically selected to be reactive under the reaction conditions used in the methods of this invention. Under these circumstances, the reaction conditions convert the selected substituent into another substituent that is either useful in an intermediate compound in the methods of this invention or is a desired substituent in a target compound.

As used herein, the term “prodrug” is intended to mean a compound that may be converted under physiological conditions or by solvolysis to a biologically active compound of the present invention. Thus, the term “prodrug” refers to a precursor of a compound of the invention that is pharmaceutically acceptable. A prodrug may be inactive or display limited activity when administered to a subject in need thereof, but is converted in vivo to an active compound of the present invention. Typically, prodrugs are transformed in vivo to yield the compound of the invention, for example, by hydrolysis in blood or other organs by enzymatic processing. The prodrug compound often offers advantages of solubility, tissue compatibility or delayed release in the subject (see, Bundgard, H., Design of Prodrugs (1985), pp. 7-9, 21-24 (Elsevier, Amsterdam). The definition of prodrug includes any covalently bonded carriers, which release the active compound of the invention in vivo when such prodrug is administered to a subject. Prodrugs of a compound of the present invention may be prepared by modifying functional groups present in the compound of the invention in such a way that the modifications are cleaved, either in routine manipulation or in vivo, to a parent compound of the invention.

### BRIEF DESCRIPTION OF THE DRAWINGS

Further aspects and advantages of the present invention will become better understood with reference to the description in association with the following in which:

FIG. 1A illustrates CD36 Western blot analysis of RPE/choroid complexes from Wistar rats (W) and SHRs (S);

FIG. 1B illustrates CD36 expression (green fluorescence) in CD36^{+/+} mice;

FIG. 1C illustrates a double-labeling with vascular marker BSA-1 (CD36 [green], BSA-1 [red], DAPI [blue]) in CD36^{+/+} mice;

FIG. 1D illustrates a Hemalun stained semi thin sections of 10-month-old CD36-deficient SHRs rats;

FIG. 1E illustrates a Hemalun stained semi thin sections of 10-month-old control Wistar (W) rats;

FIG. 1F illustrates outer nuclear layer measurements of 10-month-old Wistar rats (W; n=6) SHRs (S; n=8);

FIG. 1G illustrates a Hemalun-stained semi-thin sections (and periodic acid Schiff-stained paraffin sections [inset]) of a 1-year-old CD36^{-/-} mice;

FIG. 1H illustrates a Hemalun-stained semi-thin sections (and periodic acid Schiff-stained paraffin sections [inset]) of a age-matched WT mice;

FIG. 1I illustrates ONL thickness measurements in eyes of CD36^{-/-} (black columns; n=10) and CD36^{+/+} (white columns; n=6) mice at different ages;

FIG. 1J illustrates transmission electron microscopy of the RPE and outer segments in SHRs rats;

FIG. 1K illustrates transmission electron microscopy of the RPE and outer segments in CD36^{-/-} mice;

FIG. 1L illustrates transmission electron microscopy of the RPE and outer segments in a CD36-expressing congener strain (CD36^{+/+} mice);

FIG. 2A illustrates a micropraph of the retinal aspect of choriocapillaries in a frontal view of corrosion casts by scanning electron microscopy;

FIG. 2B illustrates a micropraph of the retinal aspect of choriocapillaries in a frontal view of corrosion casts by scanning electron microscopy. Choroidal vessels;

FIG. 2C illustrates the quantification of intercapillary space expressed as avascular area in 4-month-old Wistar rats (W) and SHRs (S) n=5 eyes/group;

FIG. 2D illustrates frontal view of the retinal aspect of choriocapillaries of 12-month-old CD36^{-/-} mice show defects in the capillary bed;

FIG. 2E illustrates frontal view of the retinal aspect of choriocapillaries of 12-month-old CD36^{+/+} mice;

FIG. 2F illustrates quantification of the avascular area over time (ages 4 months versus 12 months) of CD36^{+/+} (white columns) (n=6) and CD36^{-/-} (black columns) (n=8) mice eyes;

FIG. 2G illustrates a perpendicular view of choriocapillaries (indicated between arrows) and large choroidal vessels on cross-sectional cuts of pericentral area of CD36^{-/-} mice;

FIG. 2H illustrates a perpendicular view of choriocapillaries (indicated between arrows) and large choroidal vessels on cross-sectional cuts of pericentral area of CD36^{+/+} control mice;

FIG. 2I illustrates transmission electron microscopy of choriocapillaries of CD36^{-/-} (arrow) mice;

FIG. 2J illustrates transmission electron microscopy of choriocapillaries of CD36^{+/+} mice;

FIG. 2K illustrates quantification of capillary thickness of 12-month-old CD36^{-/-} (black column) (n=10) and CD36^{+/+} (white column) (n=8) mice eyes;

FIG. 3A illustrates RT-PCR of cDNA from primary RPE cultures from Wistar rats and SHRs;

FIG. 3B illustrates relative COX2 mRNA expression (measured by real time PCR). n=6 wells per group;

FIG. 3C illustrates COX2 immunoreactivity (green) in 4-month-old CD36^{-/-} mice; tissues were counterstained with DAPI (nuclear stain);

FIG. 3D illustrates COX2 immunoreactivity (green) in 4-month-old CD36^{+/+} mice; tissues were counterstained with DAPI (nuclear stain);

FIG. 3E illustrates VEGF immunoreactivity (green) in 4-month-old CD36^{-/-} mice; tissues were counterstained with DAPI (nuclear stain);

FIG. 3F illustrates VEGF immunoreactivity (green) in 4-month-old CD36^{+/+} mice; tissues were counterstained with DAPI (nuclear stain);

FIG. 3G illustrates relative VEGF mRNA expression (measured by real time PCR). n=6 wells per group;

FIG. 3H illustrates activation of CD36 with stimulating antibody evoked COX2 expression on RPE cell cultures from Wistar rats (W) and SHRs (measured by real time RT-PCR; n=6 wells per group;

FIG. 4A illustrates a micrograph of the retinal aspect of choriocapillaries in a frontal view of corrosion casts by scanning electron microscopy of 12-month-old COX2^{-/-} mice;

FIG. 4B illustrates a micrograph of the retinal aspect of choriocapillaries in a frontal view of corrosion casts by scanning electron microscopy of 12-month-old COX2^{+/+} mice;

FIG. 4C illustrates quantification of the avascular area (n=6 COX2^{+/+} and n=8 COX2^{-/-} eyes;

FIG. 4D illustrates cross-sectional cuts of pericentral choroidal corrosion casts of COX2^{-/-} mice;

FIG. 4E illustrates cross-sectional cuts of pericentral choroidal corrosion casts of COX2^{+/+} mice;

FIG. 4F illustrates quantification of capillary thickness of 12 month-old COX2^{+/+} and COX2^{-/-} mice (n=6 COX2^{+/+} and n=8 COX2^{-/-} eyes);

FIG. 4G illustrates relative VEGF mRNA expression (by real time RT-PCR; n=8 wells per group;

FIG. 4H illustrates VEGF expression in 4-month-old COX2^{-/-} mice;

FIG. 4I illustrates VEGF expression in 4-month-old COX2^{+/+} mice.

FIG. 5A illustrates immunolocalization of CD36 (green) in 18 week old wildtype mice. Middle panel reveals griffonia simplicifolia lectin (endothelial marker—red) and nuclear stain (DAPI—blue). Right panel reflects merging of the two images on the left.

FIG. 5B illustrates transmission electron microscopy of RPE/sub-RPE region in 52 week old wildtype and CD36−/− mice and Bruchs membrane thickness histogram.

FIG. 5C illustrates transmission electron microscopy of RPE/sub-RPE region in 18 week old wildtype and ApoE−/− mice on normal mouse chow diet (ND) or cholesterol enriched diet (Chol) treated or not with the selective CD36 ligand EP80317 (EP; from 8-18 weeks) and Bruchs membrane thickness histogram.

### DETAILED DESCRIPTION OF THE INVENTION

The invention relates to a novel method for preventing and treating dry form of AMD by activating CD36. CD36 dysfunction in vivo participate in retinal degeneration, alter the expression of essential proangiogenic factors in the RPE, or lead to neovascularization as a result of the lack of TSP-1 signaling endothelium. Spontaneous hypertensive rats (SHRs) develop visual dysfunction and retinal degeneration independent of hypertension (22, 23) as well as choroidal involution (24). These changes are secondary to invalidating CD36 mutations (25) found in (certain) SHR strains. Eyes from SHR strains bearing the invalidating CD36 mutations and from normotensive CD36^{-/-} mice were analyzed.

### Methods

Animals: CD36^{-/-} mice (26) and COX2^{-/-} mice (27) and their wild-type controls were housed at local animal facilities under 12 h light-12 h dark cycles and fed ad libitum. CD36^{-/-} mice and COX2^{-/-} mice were back-crossed on a C57B16 background for eight generations. CD36^{-/-} mice and their controls were reproduced separately thereafter. COX2^{-/-} and COX2^{+/+} mice were genotyped littermates from heterozygote genitors. Spontaneous hypertensive rats (SHRs) and Wistar controls were purchased from Janvier breeding center (Le Genest-St-Isle, France). Animal experiments were approved by the Institutional Animal Care and Use Committee of the University Paris V, Paris, France.

Western Blots: 10-day-old SHRs (n=6) and Wistar rats (n=6) were humanely killed and eyes enucleated. The eyes were dissected and RPE/choroid/sclera complexes were sonicated in ice-cold lysis buffer (Tris-HCl 50 mM [pH 6.8], 2% SDS, and 2 mM PMSF as antiprotease; the RPE is firmly attached to the choroid in the dissecting process). Protein preparation, electrophoresis, and transfer to nitrocellulose membrane were performed as previously described (29). Primary antibodies used were mouse monoclonal CD36 FA6-152 (1:500; Abcam) and with monoclonal anti-β-actin (1:5000, Santa Cruz) to control for protein loading. Proteins were revealed by corresponding secondary horseradish peroxidase-conjugated antibodies.

Immunohistochemistry: Eyes were fixed in paraformaldehyde 4% in PBS for 15 min at room temperature (RT) and rinsed in PBS before embedded in OCT (Tissue Tek). Frozen transverse sections 10 μm thick were cut and permeabilized for 10 min in 1% Triton X-100. Postfixation was performed with methanol or ethanol, depending on the antibody used. Immunolabeling with primary antibodies (1:100) rabbit polyclonal CD36 (Santa Cruz), rabbit polyclonal VEGF (Santa Cruz), rabbit polyclonal COX2 (Biomal), and endothelial cell marker Bandeiraea simplicifolia agglutinin 1 (30) (BSA-1, Sigma) was preformed overnight at RT. After washing in PBS, secondary antibodies coupled with Alexa Fluor 488 (1:100, Molecular Probes) were applied for 2 h at RT. Nuclei were labeled with DAPI (1:4000, Sigma-Aldrich) and sections were mounted with Gelmount (Biomeda). Fluorescence was observed with an Olympus BX51 microscope and photographs were taken using the same exposure times and contrast settings or a confocal microscope (Zeiss LSM 510 Laser scanning). All immunostainings were repeated at least three times, and staining without primary antibody served as negative controls.

Histology Electron Microscopy: Electron microscopy. Eyes were fixed for 1 h in 2.5% glutaraldehyde in cacodylate buffer (0.1M, pH 7.4). After 1 h, the eyeballs were dissected, fixed for another 3 h, postfixed in 1% osmium tetroxide in cacodylate buffer, and dehydrated in graduated ethanol solutions. The samples were included in epoxy resin and oriented. Semi-thin sections (1 μm), obtained with an ultramicrotome (Reichert Ultracut E [Leica]), were stained by toluidine blue, examined with light microscope, and measurements photoreceptor layer thickness were made. Ultra-thin sections (80 nm) were contrasted by uranyl acetate and lead citrate and were observed with an electron microscope JEOL 100 CX II (JOEL) with kV.

Paraffin sections: The eyes were enucleated, fixed in Bouin's fixative for 24 h, and embedded in paraffin. Sagittal sections (7 μm) were cut in parallel to the optic nerve and stained with periodic acid Schiff (PAS) and hemalun. Photoreceptor layer thickness was measured on four sections containing the optic nerve 14 μm apart from one another using digitalized images and Image J Software. The data were averaged for each eye, and the mean values from the individual eyes were statistically analyzed. Investigators performed measurements unaware of the provenance of the samples.

Reverse Transcription and Real-Time Polymerase Chain Reaction: Total RNA was isolated with RNeasy Mini Kit (Qiagen). Single-stranded cDNA was synthesized from total RNA (pretreated with DNaseI amplification grade) using oligo-dT as primer and superscript reverse transcriptase (Invitrogen). Subsequent real-time polymerase chain reaction (RT-PCR) was performed using cDNA, qPCR SuperMix-UDG Platinum SYBR Green (Invitrogen), and the following primers (0.5 μmol/μl): Actin sense, 5′-AAAGAAAGGGTGTAAAACGCAG-3 (SEQ ID NO:1); actin antisense, 5′-AAAGACCTCTATGCCAACACAG-3′ (SEQ ID NO:2); CD36 sense: 5′-GACAATCAAAAGGGAAGTTG-3′ (SEQ ID NO:3); CD36 antisense: 5′-CCTCTCTGTTTAACCTTGAT-3′ (SEQ ID NO:4); VEGF sense: 5′-TGGGATGGTCCTTGCCTC-3′ (SEQ ID NO:5); VEGF antisense: 5′-TCGCTGGAGTACACGGTGGT-3′ (SEQ ID NO:6); COX2 sense: 5′-TGCTACCATCTGGCTTCGGGAG-3′ (SEQ ID NO:7); COX2 antisense: 5′-ACCCCTCAGGTGTTGCACGT-3′ (SEQ ID NO:8). PCR reactions were performed in 40 cycles of 15 s at 95° C., 45 s at 60° C. Product was not generated in control reactions in which reverse transcriptase was omitted during cDNA synthesis.

RPE Primary Culture Ten day-old pups (Wistar rat and SHR) were humanely killed and eyes dissected and enucleated. Eyes were maintained at room temperature overnight in Dulbecco's Modified Eagle's Medium (DMEM, Invitrogen) then incubated 45 minutes with 2 mg/ml trypsin/collagenase I at 37° C. After trypsin inhibition with DMEM containing 10% fetal calf serum (FCS), the RPE layer was harvested. The RPE was plated in 12-well plates at a rate of RPE from one eye per well in DMEM containing 10% FCS, 1% penicillin/streptomycin, and 0.2% fungizone. Cells were maintained for 12 d before the phagocytosis assay.

OS isolation and phagocytosis assay and CD36 activation: OS were isolated following established protocols (31). Briefly, 20 pig's retinas were dissected and homogenized in 20% sucrose buffer, 20 mM Tris, 2 mM MgCl₂, 0.13 mM NaCl (pH 7.2). Retina samples were centrifuged on a sucrose gradient (50%, 27%) 1 h at 38,000 rpm, and OS were harvested at the ring interface and diluted in DMEM. Centrifugation 10 min at 8,000 rpm was performed and the pellet was resuspended in DMEM to obtain a stock solution of 10⁸ OS/ml.

Confluent RPE monolayers were challenged for 1 h with 150 μl of ROS, and then 850 μl of complete medium was added. Cells were washed and 350 μl of RLT lysis buffer (RNeasy mini kit, Qiagen) was added for RNA extraction at 0 and 6 h.

The CD36 antibody FA6-152 antibody (Abeam) can activate CD36 by dimerization of the receptor as previously shown (16). CD36 was activated with 20 μg/ml of FA6-152 antibody as described (16). RPE cells from Wistar rats or SHRs were incubated with either FA6-152 or control antibody in DMEM for 4 h and mRNA was achieved by a 30 min preincubation with 10⁻⁶ M DUP697; this concentration was maintained throughout the experiments.

Vascular corrosion casts: Animals were killed by CO₂ inhalation followed by a thoracotomy. Venous catheter was introduced into the aorta through the left heart ventricule, and the right auricle was cut to allow evacuation of injected products. A perfusion was performed with a mixture of red Mercox resin and catalyst (Ladd Research). Eyes were extracted and lenses were removed. Tissues were conserved overnight at 37° C. in PBS to allow complete polymerization, and then digested by 5% KOH for 2 wk at 37° C. until only the vascular corrosion casts remained. Distilled water was used to remove salt and the mold was dried. Only corrosion casts with completely filled iris vessels were used, to exclude corrosion casts from incomplete perfusion. Retinal vasculature was removed using forceps. The specimens were mounted on SEM stubs, coated with gold palladium, and scanned at an accelerating voltage of 117 kV. In order to measure the thickness of the choriocapillary lumen, corrosion casts were cut paracentrally (1 mm from the aperture of the optic nerve) and positioned for perpendicular views of the choriocapillaries. To analyze the intercapillary space (avascular area), the casts were positioned for frontal views of the choriocapillaries. Electron micrographs were scanned and analyzed using Image J Software (http://rsb.info.nih.gov/ij). The avascular area was measured on frontal views and expressed as the percentage of intercapillary surface (space between the plastic capillary casts) of the whole area. Thickness of choriocapillaries was measured on perpendicular views of the cast from the retinal to scleral side of the choriocapillary cast.

Statistical analysis: Variance was analyzed by Kruskal-Wallis test. Data between two groups were compared with nonparametric Mann Whitney U-test. All analysis and graphic representation were performed with Prism software (version 4.0c; GraphPad Software), and values are represented as mean±standard error of the mean (SEM). P values were calculated for a confidence interval of 95% and P values of less than 0.05 were considered significant.

### Results

Retinal degeneration in CD36-deficient animals: In vitro participation of CD36 in the phagocytosis of OS by human (15) and rat RPE cells in vitro (16) has previously been described. However, the involvement of CD36 in phagocytosis in vivo has been unknown. To assess the role of CD36 in phagocytosis and retinal homeostasis in vivo, two animal models: an albino SHR strain containing several CD36 mutations that lead to undetectable levels of CD36 expression in several tissues (24), and pigmented normotensive CD36^{-/-} mice (32) were examined.

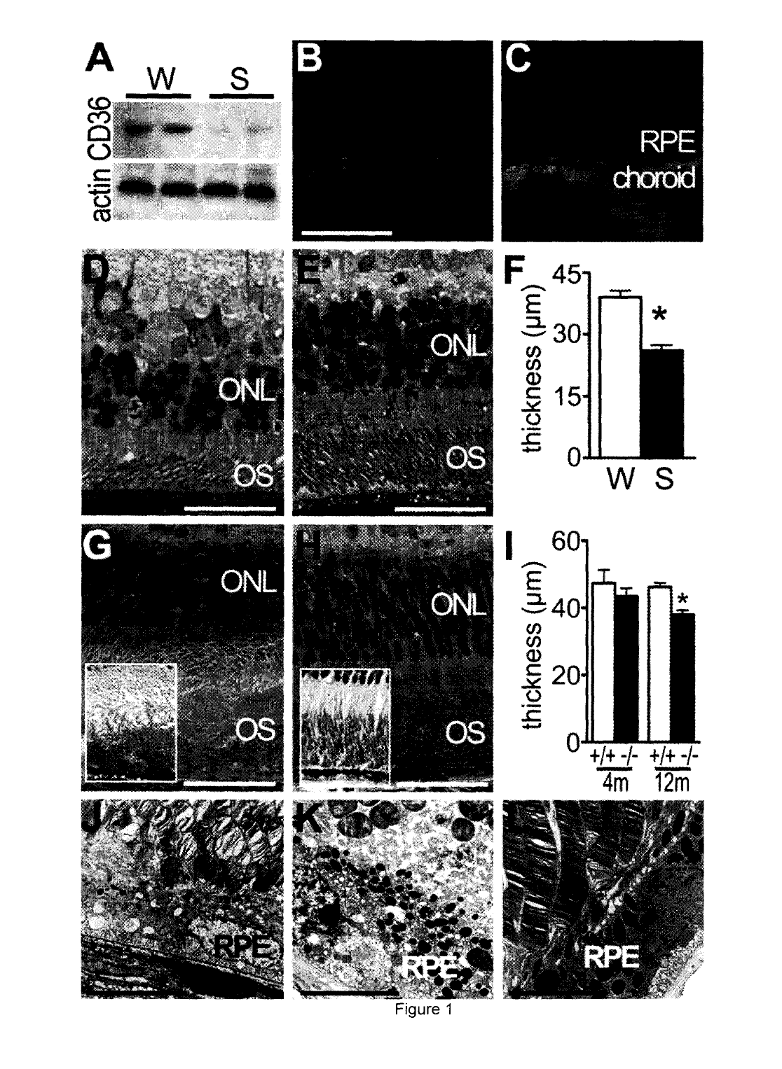

Western blot analysis of RPE/choroid complexes showed greatly diminished CD36 protein expression in the eyes of SHRs compared to the Wistar rat control strain in vivo (FIG. 1A) (n=4 eyes per group). CD36 localization in mice was analyzed using specific antibodies. CD36 was expressed in the basal aspect of the RPE (FIG. 1B) and in choroidal vessels.

Semi-thin sections of 10-month-old SHRs (FIG. 1D) revealed an irregular outer nuclear layer (ONL) and patchy retinal degeneration. OS morphology was disrupted compared to the regularly shaped photoreceptors of control animals (FIG. 1E).

Quantification of the ONL thickness revealed a significant 26% reduction in ONL at this stage (FIG. 1F; *p<0.0001). Similarly, semi-thin sections of 12-month-old CD36^{-/-} mice (FIG. 1G) showed a thinned irregular outer nuclear layer (ONL) and ill-defined limit between inner and outer photoreceptor segments; OS were irregular inner and accumulated in some areas as seen in periodic acid Schiff stains (FIG. 1G inset). Whereas 12-month-old CD36^{+/+} mice showed regularly shaped photoreceptors (FIG. 1H). Although regional differences in degenerative changes were observed, overall ONL was 17% thinner (p<0.05) at 12 months of age; this was not yet observed at 4 months of age (FIG. 1I; *p=0.0095 significant difference at 12 months).

Detailed morphological evaluation by electron microscopy (EM) of 10-month-old SHRs (FIG. 1J) and 12-month-old CD36^{-/-} mice (FIG. 1K) showed OS detachment from the RPE villi. Strikingly, OS appeared in oblique as well as in perpendicular planes of sagittal sections of the eyes (note FIGS. 1J and 1K sagittal RPE choroids section plane). In control animals (FIG. 1L, 12-month-old CD36^{+/+} mouse), OS were in tight contact with RPE villi and were longitudinal or slightly oblique throughout the sagittal eye sections; perpendicularly (cross)-sectioned OS were never observed in control animals. These differences might be due to OS overgrowth in CD36 deficiency secondary to RPE phagocytosis deficiency. The elongated OS in these animals could explain the accumulation of periodic acid Schiff material (FIG. 1G inset) and the disorientation of the OS (on EM; FIGS. 1J and 1K). Results are representative of at least three independent experiments; scale bar: FIGS. 1B, 1C, 1D, 1E, 1G and 1H=50 μm and FIGS. 1J-L=5 μm.

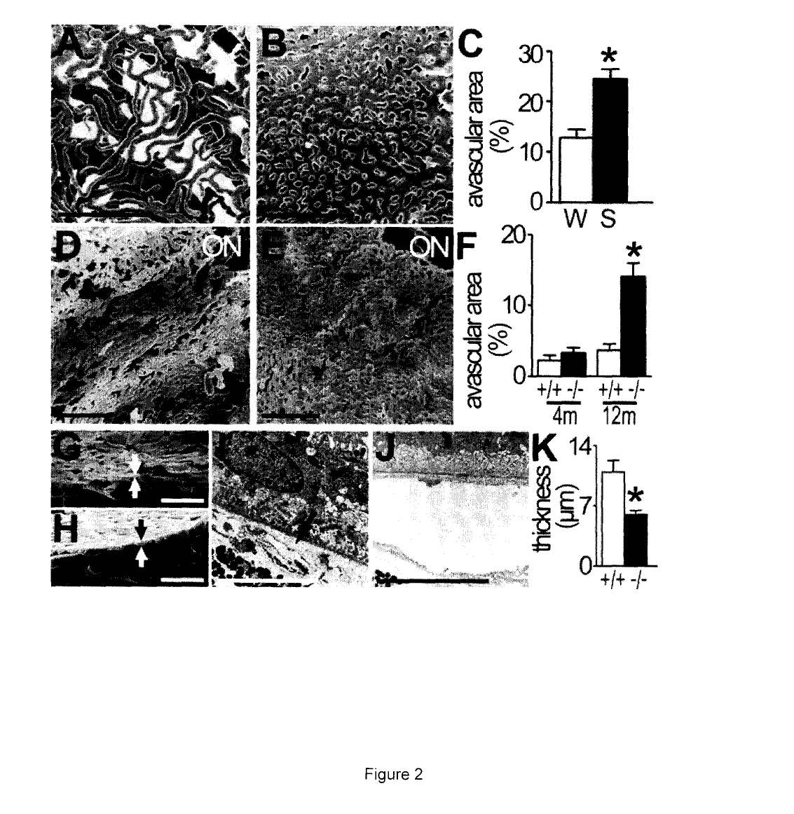

Choridal involution in CD-36-deficient animals: Choroidal involution is a main feature of dry AMD (4). Little is known of the molecular mechanisms leading to choroidal involution. Interestingly, choroidal involution was reported in SHRs several decades ago (25), prior to knowledge of their CD36 status. To study the influence of CD36 deficiency on choroidal integrity, choroids of CD36-deficient SHRs and CD36^{-/-} mice were analyzed. Vascular corrosion casts of the choriocapillaries of 4-month-old SHRs revealed a vascular rarefaction of the choriocapillaries (FIG. 2A; choroidal vessels [darker grey] can be seen through the intercapillary spaces of the choriocapillaries in 4-month-old SHRs rats) compared to age matched control Wistar rats (FIG. 2B; darker grey cannot be seen through the intercapillary spaces of the choriocapillaries in age-matched Wistar rats). Correspondingly, quantification of intercapillary space revealed a significant increase in avascular area in SHRs (FIG. 2C; *p=0.0027). Similarly, choriocapillaries of 12-month-old CD36^{-/-} mice showed capillary dropout and a moth-eaten appearance (FIG. 2D) compared to the dense microvasculature of CD36^{+/+} mice (FIG. 2E); this was reflected by an increase in avascular area of choroids of 12-month-old mice CD36^{-/-} mice compared to CD36^{+/+} mice; a tendency toward an increase in avascularity was already detected by 4 months of age, although this was not yet statistically significant (FIG. 2F; *p=0.0286 significant difference at 12 months). In addition, cross-sectional views of the vascular corrosion casts showed severe thinning of choroids from CD36^{-/-} (FIG. 2G) compared to control mice (FIG. 2H). This involution was also observed using transmission electron microscopy, where choriocapillaries of CD36^{-/-} mice were either missing or exhibited severely diminished thickness compared to those of CD36^{+/+} mice (FIG. 2I-2K) (2K, *p=0.0062). In contrast, capillary density of other organs such as the skin, brain, and ocular muscles were not affected. Results are representative of at least three independent experiments. Scale bar, FIGS. 2A, 2B, 2D, 2E, 2G and 2H=100 μm; and FIGS. 2I and 2J=5 μm.

OS-induced COX2 and VEGF expression in RPE is CD36-dependent: In vivo RPE cells express prosurvival/proangiogenic factors such as COX2 that may be necessary for choriocapillary integrity (33) upon OS stimulation (19). The COX2 expression in the RPE appears to be phagocytosis dependent, as RPE primary cell cultures express COX2 once stimulated with OS (19). Since CD36 influences phagocytic activity in vitro (16), the influences of CD36 on the expression of COX2 in RPE was analyzed.

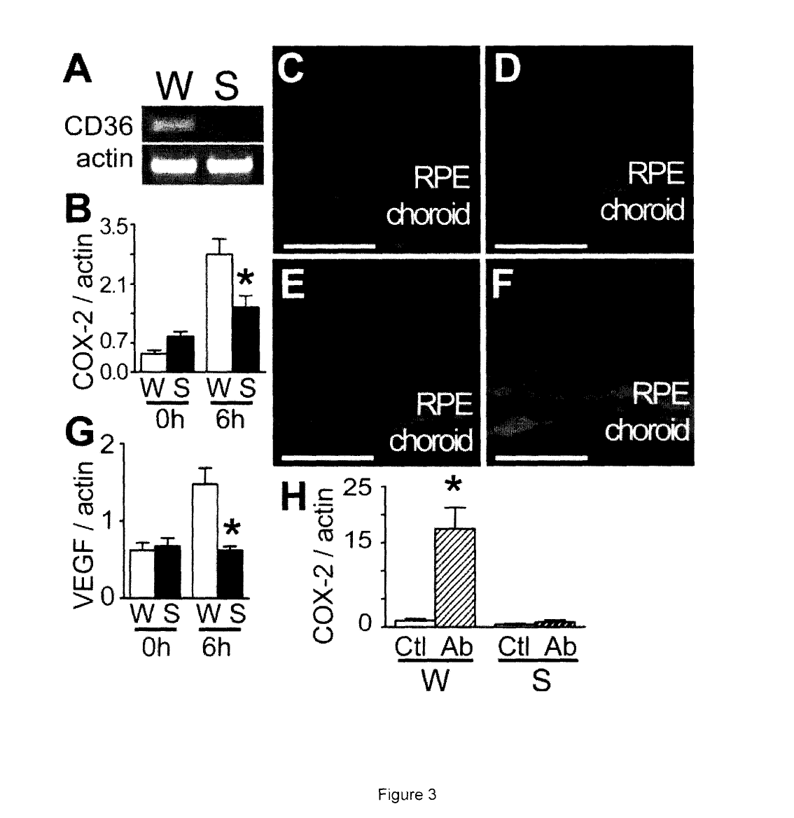

Absence of CD36 mRNA in the SHR strain was verified by RT-PCR on primary RPE cells (FIG. 3A). Primary RPE cell cultures from CD36-deficient SHRs and control Wistar rats were exposed to OS for different durations, and COX2 mRNA analyzed (by real-time PCR). Control RPE cells exhibited significant increases in COX2 mRNA expression 6 h after stimulation (FIG. 3B; *p=0.0152 significant difference between control and CD36-deficient rats at 6 h exposed in culture to rod outer segments) as previously described (19). In contrast, rat primary RPE cells deficient in CD36 (FIG. 1A) failed to respond to OS exposure (FIG. 3B). Analogously, in the eyes of CD36^{-/-} mice COX2 immunoreactivity was reduced in the RPE (FIG. 3C) compared to that of CD36^{+/+} mice (FIG. 3D). Moreover, direct activation of CD36 even in the absence of OS, using a CD36 antibody at stimulating concentration (16), greatly induced COX2 in RPE from CD36-expressing Wistar rats, but not in RPE from the CD36-deficient SHR strain (FIG. 3H; *p=0.0012, COX2 expression significantly different between control [Ctl] and antibody-treated [Ab] Wistar RPE culture).

Because COX2 activity can control VEGF expression (20) and VEGF expression in RPE is essential for normal choroidal development (34) and possibly its homeostasis in rodents, CD36 expression also affecting VEGF was tested. VEGF mRNA expression in RPE was also positively regulated by phagocytosis and blunted by CD36 deficiency (FIG. 3G; *p=0.0087 significant difference at 6 h in RPE cells of Wistar [W] and SHR [S] rats exposed in culture to rod outer segments); likewise, RPE of CD36^{-/-} mice expressed diminished VEGF immunoreactivity (FIG. 3E) compared to CD36^{+/+} mice in vivo (FIG. 3F). Results are representative of at least three independent experiments. Ab, CD36 antibody FA6-152. Scale bar for FIGS. 3=50 μm. Photographs of immunohistochemical signal were taken with identical parameters in CD36^{-/-} and CD36^{+/+} mice.

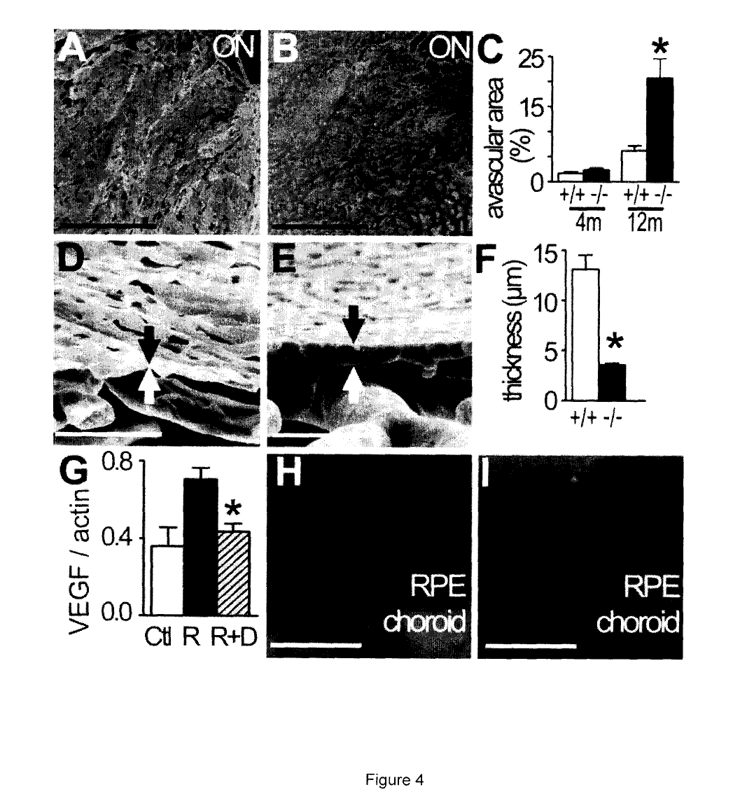

COX2^{-/-} mice develop choroidal involution: On the basis of observations presented above, the expression of COX2 in RPE affecting or not choroidal homeostasis was next investigated. The choroidal morphology in COX2−/− mice and their wild-type congeners was investigated. Compared to COX2^{+/+} mice (FIG. 4B), vascular corrosion casts of 12-month-old COX2^{-/-} mice (FIG. 4A) were extremely brittle and showed a moth-eaten appearance secondary to capillary dropout as detected by the increased avascular area; these changes were not yet perceptible in 4-month-old mice (FIG. 4C; *p=0.007, COX2^{-/-} significantly different from COX2^{+/+} at 12 months). Cross-sectional views revealed severe involution of the choriocapillaries in COX2^{-/-} (FIG. 4D) relative to wild-type mice (FIG. 4E), as noted by the significant reduction in capillary thickness in COX2^{-/-} mice at 12 months of age (FIG. 4F *p=0.0007). As seen in CD36^{-/-} mice, capillary morphometry in other organs (skin, brain, and ocular muscles) were not altered (unpublished data). Interestingly, in contrast to CD36^{-/-} mice, COX2^{-/-} mice did not develop degeneration of photoreceptors by 12 months of age (ONL thickness COX2^{+/+}±SEM [n=4]=101±5.6 μm; COX2^{-/-} [n=4]=109±9.8 μm).

As mentioned above, COX2 activity can regulate VEGF expression in various cells (20). COX2 activity influencing VEGF expression in OS-exposed primary rat RPE cultures was next investigated. Indeed, selective COX2 inhibition by DUP697 (29) prevented OS-induced VEGF mRNA expression (FIG. 4G *p=0.0029 rod outer segments with DUP697 [R+D] significantly different from rod outer segments alone [R] in primary RPE culture of Wistar rats [Ctl, white column], exposed to rod outer segments in absence [R, black column] or presence of the COX2 inhibitor DUP697 [10⁻⁶ M] [R+D]), but not basal VEGF expression. Similar observations were made in vivo, whereby VEGF expression in RPE was substantially reduced in COX2^{+/+} mice (FIG. 4I). Altogether, the consequences of COX2 deficiency on VEGF expression in RPE and in turn on choroidal integrity are nearly identical to those observed in CD36-deficient animals (FIG. 2). Results are representative of at least three independent experiments. Scale bar for FIGS. 4=100 μm. Photomicrographs of immunohistochemical signal were taken with identical parameters.

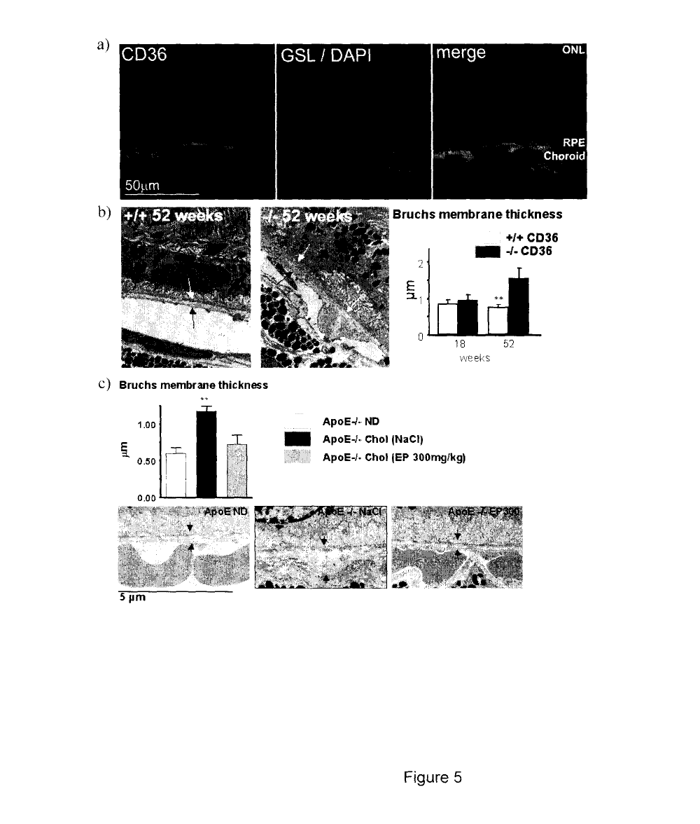

Note the localization of CD36 on RPE and choriocapillarie on FIG. 5A. ONL refers to outer nuclear layer. The patchy BM thickening in CD36−/− mice is illustrates on FIG. 5B by electron; microscopy. As demonstrated on FIG. 5B, CD36^{+/+} 52 week old mice show significant thinner Bruchs Membrane (BM) than the CD36^{-/-} 52 week old mice (the histogram is representative of a compilation of 3 animals in each group). Transmission electron microscopy of RPE/sub-RPE region in 18 week old wildtype and ApoE^{-/-} mice on normal mouse chow diet (ND) or cholesterol enriched diet (Chol) treated or not with the selective CD36 ligand EP80317 (EP; from 8-18 weeks) is shown on FIG. 5C. BM is the region between arrows. Note BM thickening in ApoE^{-/-} mice on atherogenic diet, while those treated with EP80317 are not significantly different from ApoE−/− on normal diet which also resemble wildtype (data not shown) (the histogram is representative of a compilation of 3 animals in each group).

Discussion

Retinal degeneration and choroidal involution are cardinal features of the predominant, “dry” form of AMD. The Applicant demonstrate that CD36 deficiency causes photoreceptor/OS degeneration and choroidal involution in rats and mice. Those results also show that CD36 expression is necessary for OS induced prosurvival/proangiogenic COX2 expression in RPE in vitro and that COX2 ablation causes similar choroidal involution in vivo. Therefore, the link between photoreceptor degeneration and choroidal involution, the main features of dry AMD is demonstrated in this application.

CD36 was expressed in mice in the basal aspect of the RPE and in choroidal vessels, as described in rat and human (15). In contrast to data reported from in vitro experiments (16), CD36 does not seem to be essential for basal RPE phagocytosis in vivo, since an absolute defect in RPE phagocytosis would lead to a more rapid and complete retinal degeneration (8), whereas CD36 deficiency is associated with late-onset retinal degeneration. It has been suggested that CD36 plays a predominant role in OS phagocytosis mainly under oxidative conditions (17). Interestingly, the relatively late morphological alterations observed in CD36-deficient animals seem to coincide with an increase in oxidative stress, as antioxidant defenses diminish with age (35). This inference is reinforced by the accrued OS degeneration observed in oxidative stress-prone albino SHRs compared to pigmented CD36^{-/-} mice (FIG. 1).

Choroidal involution was reported in SHRs several decades ago (25), prior to knowledge of their CD36 status. The present findings confirm the choroidal vascular rarefaction described in SHRs (25). Furthermore, experiments using normotensive CD36-deficient animals (32) show that this rarefaction occurs independently of hypertension and is secondary to CD36 deficiency. The deficient antiangiogenic signaling in the vascular endothelium due to the suppression of CD36 as the main receptor of TSP-1 (21) does not significantly counterbalance this effect.

The normal appearance of capillary beds distant from the RPE, show a predominant role of local paracrine factors in CD36 dependent choroidal involution. In vivo, these CD36-dependent paracrine factors likely originate from the CD36-expressing RPE cells adjacent to the choriocapillaries. RPE cells express prosurvival/proangiogenic factors such as COX2 in vivo (33) that is necessary for choriocapillary integrity. COX2 expression in the RPE is significantly augmented by retinal OS phagocytosis (19). The present study show that CD36 expression in RPE is necessary for the OS-induced expression of COX2 in RPE primary cultures, as CD36 deficiency blunted the OS response in vitro and diminished COX2 expression in vivo.

CD36 activation sufficed to induce COX2, demonstrating that OS-induced COX2 expression in RPE cells is directly mediated by CD36 as recently described for oxidized low density lipoprotein (oxLDL) in COX2 expression in macrophages (36). In macrophages, CD36 stimulation has been shown to activate the transcription factor nuclear factor kappa B (NF-KB) (37), which controls COX2 expression (38), and similar mechanism might be involved in the RPE. Taken together, these results show that CD36 exerts an important permissive role in evoking the expression of prosurvival/proangiogenic factor COX2 in the RPE. Choroidal involution is at least in part due to the observed COX2 down-regulation in RPE, since in the present study COX2 deletion led to a similar choroidal involution. This inference is further substantiated by the interplay between COX2 and another major prosurvival/proangiogenic factor, VEGF, such that a COX2 activity deficiency (genetic and pharmacological) depressed VEGF immunoreactivity in RPE in vitro and in vivo, as seen in mice deficient in CD36, which itself also regulates both COX2 and VEGF expression. Together these findings demonstrate that diminished expression of CD36-dependent COX2 and VEGF in RPE contribute to the rarefaction of the adjacent choriocapillaries.

The present study shows a novel molecular mechanism of photoreceptor degeneration and choroidal rarefaction, key cardinal features of dry AMD. Furthermore these findings demonstrate that pharmacological activation of CD36 or restoration of CD36 expression in RPE of patients with dry AMD can be used therapeutically to prevent photoreceptor cell death by boosting SE renewal and to maintain a healthy choroid and retinal oxygenation by enhanced COX2 expression. Therefore administering CD36 activator compounds to a subject would prevent or treat dry AMD.

A class of azapeptide compounds has been discovered, which are analogs of growth-releasing peptide-6 (GHRP-6). The compounds selectively bind to CD36 with loss of binding activity at the ghrelin receptor GHS-R1a. The compounds can be used to prevent and treat dry AMD. The azapeptide is the following Formula I:

A-(Xaa)ₐ-N(R^{A})—N(R^{B})—C(O)-(Xaa′)_{b}-B I

wherein

a is an integer from 0 to 5;

b is an integer from 0 to 5;

Xaa and Xaa are each any D or L amino acid residue or a D,L amino acid residue mixture;

A is

1) H,

2) C₁-C₆ alkyl,

3) C₂-C₆ alkenyl,

4) C₂-C₄ alkynyl,

5) C₃-C₇ cycloalkyl,

6) haloalkyl,

7) heteroalkyl,

8) aryl,

9) heteroaryl,

10) heteroalkyl,

11) heterocyclyl,

12) heterobicyclyl,

13) C(O)R³,

14) SO₂R³,

15) C(O)OR³, or

16) C(O)NR⁴R⁵,

wherein the alkyl, the alkenyl, the alkynyl and the cycloalkyl are optionally substituted with one or more R¹ substituents; and wherein the aryl, the heteroaryl, the heterocyclyl and the heterobicyclyl are optionally substituted with one or more R² substituents;

B is

1) OH,

2) OR³, or

3) NR⁴R⁵;

R^{A} and R^{B} are independently chosen from

1) H,

2) C₁-C₆ alkyl,

3) C₂-C₆ alkenyl,

4) C₂-C₆ alkynyl,

5) C₃-C₇ cycloalkyl,

6) C₅-C₇ cycloalkenyl,

7) haloalkyl,

8) heteroalkyl,

9) aryl,

10) heteroaryl,

11) heterobicyclyl, or

12) heterocyclyl,

wherein the alkyl, alkenyl, alkynyl and the cycloalkyl and cycloalkenyl are optionally substituted with one or more R¹ substituents; and wherein the aryl, the heteroaryl, the heterocyclyl and the heterobicyclyl are optionally substituted with one or more R² substituents,

or alternatively, R^{A} and R^{B} together with the nitrogen to which each is bonded form a heterocyclic or a heterobicyclic ring;

R¹ is

1) halogen,

2) NO₂,

3) CN,

4) haloalkyl,

5) C₃-C₇ cycloalkyl,

6) aryl,

7) heteroaryl,

8) heterocyclyl,

9) heterobicyclyl,

10) OR⁶,

11) S(O)₂R³,

12) NR⁴R⁵,

13) NR⁴S(O)₂R³,

14) COR^{E},

15) C(O)OR⁶,

16) CONR⁴R⁵,

17) S(O)₂NR⁴R⁵,

18) OC(O)R⁶,

19) SC(O)R³,

20) NR⁶C(O)NR⁴R⁵,

21) heteroalkyl,

22) NR⁶C(NR⁶)NR⁴R⁵, or

23) C(NR⁶)NR⁴R⁵;

wherein the aryl, heteroaryl, heterocyclyl, and heterobicyclyl are optionally substituted with one or more R² substituents;

R² is

1) halogen,

2) NO₂,

3) CN,

4) C₁-C₆ alkyl,

5) C₂-C₆ alkenyl,

6) C₂-C₄ alkynyl,

7) C₃-C₇ cycloalkyl,

8) haloalkyl,

9) OR⁶,

10) NR⁴R⁵,

11) SR⁶,

12) COR^{E},

13) C(O)OR⁶,

14) S(O)₂R³,

15) CONR⁴R⁵,

16) S(O)₂NR⁴R⁵,

17) aryl,

18) heteroaryl,

19) heterocyclyl,

20) heterobicyclyl,

21) heteroalkyl,

22) NR⁶C(NR⁶)NR⁴R⁵, or

23) C(NR⁶)NR⁴R⁵,

wherein the aryl, the heteroaryl, the heterocyclyl, and the heterobicyclyl are optionally substituted with one or more R⁷ substituents;

R³ is

1) C₁-C₆ alkyl,

2) C₂-C₆ alkenyl,

3) C₂-C₄ alkynyl,

4) C₃-C₇ cycloalkyl,

5) haloalkyl,

6) aryl,

7) heteroaryl,

8) heterocyclyl, or

9) heterobicyclyl,

wherein the alkyl, the alkenyl, the alkynyl and the cycloalkyl are optionally substituted with one or more R¹ substituents; and wherein the aryl, the heteroaryl, the heterocyclyl and the heterobicyclyl are optionally substituted with one or more R² substituents;

R⁴ and R⁵ are independently chosen from

1) H,

2) C₁-C₆ alkyl,

3) C₂-C₆ alkenyl,

4) C₂-C₆ alkynyl,

5) aryl,

6) heteroaryl, or

7) heterocyclyl,

or R⁴ and R⁵ together with the nitrogen to which they are bonded form a heterocyclic ring;

R⁶ is

1) H,

2) C₁-C₆ alkyl,

3) C₂-C₆ alkenyl,

4) C₂-C₆ alkynyl,

5) aryl,

6) heteroaryl, or

7) heterocyclyl;

R⁷ is

1) halogen,

2) NO₂,

3) CN,

4) C₁-C₆ alkyl,

5) C₂-C₆ alkenyl,

6) C₂-Cₐ alkynyl,

7) C₃-C₇ cycloalkyl,

8) haloalkyl,

9) OR⁶,

10) NR⁴R⁵,

11) SR⁶,

12) COR⁶,

13) C(O)OR⁶,

4) S(O)₂R³,

15) CONR⁴R⁵,

16) S(O)₂NR⁴R⁵,

17) heteroalkyl,

18) NR⁶C(NR⁶)NR⁴R⁵, or

19) C(NR⁶)NR⁴R⁵;

or a salt thereof, or a prodrug thereof;

wherein the following compounds are excluded:

### A is H, (Xaa)ₐ is His-D-Trp, R^{A} is H, R^{B} is CH₃, (Xaa′)_{b} is Trp-D-Phe-Lys and B is NH₂;

### A is H, (Xaa)ₐ is His-D-Trp-Ala-Trp, R^{A} is H, R^{B} is CH₂Ph, (Xaa′)_{b} is Lys and B is NH₂;

A is H, (Xaa)ₐ is (D/L)-His, R^{A} is H, R^{B} is CH₂-p-C₆H₄OH, (Xaa′)_{b} is Ala-Trp-D-Phe-Lys and B is NH₂;
A is H, (Xaa)ₐ is His-D-Trp-Ala, R^{A} is H, R^{B} is CH₂-p-C₆H₄OH, (Xaa′)_{b} is D-Phe-Lys and B is NH₂; and

### A is H, (Xaa)ₐ is His-D-Trp-Ala-D-Phe, R^{A} is H, R^{B} is (CH₂)₄NH_{2, b} is 0, and B is NH₂.

### REFERENCES

1. Friedman D S, O'Colmain B J, Munoz B, Tomany S C, McCarty C et al. Prevalence of age-related macular degeneration in the United States. Arch Ophtalmol: 2004:122:564-572.
2. De Jong P. Age related macular degeneration. New Engl. J. Med. 2006:355:1474-1485.
3. Rattner A, Nathans J. Macular degeneration: recent advances and therapeutic opportunities. Nature Reviews Neuroscienc. 2006:7:860-872.
4. Sarks S H. Ageing and degeneration in the macular region: a clinico-pathological study. Brit. Ophtalmol. 1976:60:324-341.
5. Friedman E. The role of the atherosclerotic process in the pathogenesis of age-related macular degeneration. Am. J. Ophtalmol. 2000:130:658-663.
6. Vingerling J R, Dielemans I, Bots M I et al. Age-related macular degeneration is associated with atherosclerosis. The Rotterdam study. Am. J. Epidemiol. 1995:142:404-409.
7. Green W R, Enger C. Age-related macular degeneration histopathological studies. The 1992 Lorenz E. Zimmerman Lecture. Ophtalmology. 1993:100:1519-1535.
8. Lee P, Wang C C, Adamis A P. Ocular neovascularization: an epidemiologic review. Surv. Ophtalmol. 1998:43:245-269.
9. Young R W, Bok D. Participation of the retinal pigment epithelium in the rod outer segment renewal process. J. Cell. Biol. 1969:42:392-403.
10. Edwards R B, Szamier R B. Defective phagocytosis of isolated rod outer segments by RCS rat retinal pigment epithelium in culture. Science 1977:197:1001-1003.
11. Nandrot E, Dufour E M, Provost A C, Pequignot M O, Bonnel S et al. Homozygous deletion in the coding sequence of the c-mer gene in RCS rats unravels general mechanisms of physiological cell adhesion and apoptosis. Neurobiol. Dis. 2000:7:586-599.
12. D'Cruz P M, Yasumura D, Weir J, Matthes M T, Abderrahim H et al. Mutation of the receptor tyrosine kinase gene Mertk in the retinal dystrophic RCS rat. Hum. Mol. Genet. 2000:9:645-651.
13. Finnemann S C, Bonilta V L, Marmorstein A D, Rodriguez-Boulan E. Phagocytosis of rod outer segments by retinal pigment epithelial cells requires alpha (v)beta 5 integrin for binding but not for internalization. Proc. Natl. Acad. Sci. USA. 1997:94:12932-12937.
14. Endemann G, Stanton L W, Madden K S, Bryant C M, White R T et al. CD36 is a receptor for oxidized low density lipoprotein. J. Biol. Chem. 1993:268:11811-11816.
15. Ryeom S W, Sparrow J R, Silverstein R L. CD36 participates in the phagocytosis of rod outer segments by retinal pigment epithelium. J. Cell. Sci. 1996:109:387-395.
16. Finnemann S C, Silverstein R L. Differential roles of CD36 and alphavbeta5 integrin in photoreceptor phagocytosis by the retinal pigment epithelium. J. Exp. Med. 2001: 194: 1289-1298.
17. Sun M, Finnemann S C, Febbraio M, Shan L, Annangudi S P et al. Light-induced oxidation of photoreceptor outer segment phospholipids generates ligands for CD36-mediated phagocytosis by retinal pigment epithelium: a potential mechanism for modulating outer segment phagocytosis under oxidant stress conditions. J. Biol. Chem. 2006:281:4222-4230.
18. Chowers I, Kim Y, Farkas R H, Gunatilaka T L, Hackam A S et al. Changes in retinal pigment epithelial gene expression induced by rod outer segment uptake. Invest. Ophthalmol. Vis. Sci. 2004:45:2098-2106.
19. Ershov A V, Bazan N G. Induction of cyclooxygenase-2 gene expression in retinal pigment epithelium cells by photoreceptor rod outer segment phagocytosis and growth factors. J. Neurocsi. Res. 1999:58:254-261.
20. Tsujii M, Kawano S, Tsuji S, Sawaoka H, Hori M. et al. Cyclooxygenase regulates angiogenesis induced by colon cancer cells. Cell 1998:93:705-716.
21. Jimenez B, Volpert O V, Crawford S E, Febbraio M, Silverstein R L et al. Signals leading to apoptosis-dependent inhibition of neovascularization by thombospondin-1. Nat. Med. 2000:6:41-48.
22. Rogers L J, Bolden S W, Patrech A S, Ehrlich D. Visual dysfunction in the spontaneously hypertensive rat. Physiol Behay. 1993:54:903-907.
23. Li S, Lam T T, Fu J, Tso M O. Systemic hypertension exaggerates retinal photic injury. Arch Ophthalmol. 1995:113:521-526.
24. Aitman T J, Glazier A M, Wallace C A, Cooper L D, Norsworthy P J et al. Identification of Cd36 (Fat) as an insulin-resistance gene causing defective fatty acid and glucose metabolism in hypertensive rats. Nat. Genet. 1999:21:76-83.
25. Funk R, Rohen J W. Comparative morphological studies on blood vessels in eyes of normotensive and spontaneously hypertensive rats. Exp. Eye Res. 1989:40:191-203.
27. Febbraio M, Abumrad N A, Hajjar D P, Sharma K, Cheng W et al. A null mutation in murine CD36 reveals an important role in fatty acid and lipoprotein metabolism. J. Biol. Chem. 1999:274:19055-19062.
28. Morpham S G, Langenbach R, Loftin C D, Tiano H F, Vouloumanos N et al. Prostaglandin synthase 2 gene disruption causes severe renal pathology in the mouse. Cell 1995:83:473-482.
29. Kargman S, Wong E, Greig G M, Falgueyret J P, Cromlish W et al. Mechanism of selective inhibition of human prostaglandin G/H synthase-1 and -2 in intact cells. Biochem. Pharmacol. 1996:52:1113-1125.
30. Sahagun G, Moore S A, Fabry Z, Schelper R L, Hart M N. Purification of murine endothelial cell cultures by flow cytometry using fluorescein-labeled griffonia simplicifolia agglutinin. Am. J. Pathol. 1989:134:1227-1232.
31. Molday R S, Hicks D, Molday L. Peripherin. A rim-specific membrane protein of rod outer segment discs. Invest. Ophthalmol. Vis. Sci. 1987:28:1227-1232.
32. Kincer J F, Uittenbogaard A, Dressman J, Guerin T M, Febbraio M et al. Hypercholesterolemia promotes a CD36-dependent and endothelial nitric-oxide synthase-mediated vascular dysfunction. J. Biol. Chem. 2002:277:23525-23533.
33. Sennlaub F, Valamanesh F, Vazquez-Tello A, El-Asrar A M, Checchin D et al. Cyclooxygenase-2 in human and experimental ischemic proliferative retinopathy. Circulation 2003:108:198-204.
34. Marneros A G, Fan J, Yokoyama Y, Gerber H P, Ferrara N et al. Vascular endothelial growth factor expression in the retinal pigment epithelium is essential for choriocapillaries development and visual function. Am. J. Pathol. 2005:167:1451-1459.
35. Harman D. Aging: a theory based on free radical and radiation chemistry. J. Gerontol. 1956:11:298-300.
36. Kanayama M, Yamaguchi S, Shibata T, Shibata N, Kobayashi M et al. Identification of a serum component that regulates cyclooxygenase-2 gene expression in cooperation with 4-hydroxy-2-nonenal. J. Biol. Chem. 2007:282:24166-24174.
37. Janabi M, Yamashita S, Hirano K, Sakai N, Hiraoka H et al. Oxidized LDL-induced NF-kappa B activation and subsequent expression of proinflammatory genes defective in monocyte-derived macrophages from CD36-deficient patients. Arterioscler. Thromb. Vasc. Biol. 2000:20:1953-1960.
38. Wu D, Marko M, Claycombe K, Paulson K E, Meydani S N. Ceramide-induced and age-associated increase in macrophage COX-2 expression is mediated through up-regulation of NF-kappa B activity. J. Biol. Chem. 2003:278:10983-10992.

1. A method for preventing or treating dry age-related macular degeneration in a subject comprising administering a therapeutically effective amount of a CD36 activator compound to said subject.

2. The method according to claim 1 wherein said subject is a human or a non-human mammal.

3. The method according to claim 2 wherein the non-human mammal is selected from the group consisting of cats, dogs, swine, cattle, sheep, goats, horses, rabbits, rats and mice.

4. The method according to claim 45 wherein said CD36 activator antibody is CD36 antibody FA6-152.

5. The method according to claim 46 wherein said growth hormone-releasing peptide derivative is EP80317.

6. The method according to claim 1 wherein said CD36 activator compound is an azapeptide compound of Formula I:
A-(Xaa)ₐ-N(R^{A})—N(R^{B})—C(O)-(Xaa′)_{b}-B
wherein
a is an integer from 0 to 5;
b is an integer from 0 to 5;
Xaa and Xaa′ are each any D or L amino acid residue or a D,L amino acid residue mixture;
A is
1) H,
2) C₁-C₆ alkyl,
3) C₂-C₆ alkenyl,
4) C₂-C₄ alkynyl,
5) C₃-C₇ cycloalkyl,
6) haloalkyl,
7) heteroalkyl,
8) aryl,
9) heteroaryl,
10) heteroalkyl,
11) heterocyclyl,
12) heterobicyclyl,
13) C(O)R³,
14) SO₂R³,
15) C(O)OR³, or
16) C(O)NR⁴R⁵,
wherein the alkyl, the alkenyl, the alkynyl and the cycloalkyl are optionally substituted with one or more R¹ substituents; and wherein the aryl, the heteroaryl, the heterocyclyl and the heterobicyclyl are optionally substituted with one or more R² substituents;
B is
1) OH,
2) OR³, or
3) NR⁴R⁵;
R^{A} and R^{B} are independently chosen from
1) H,
2) C₁-C₆ alkyl,
3) C₂-C₆ alkenyl,
4) C₂-C₆ alkynyl,
5) C₃-C₇ cycloalkyl,
6) C₅-C₇ cycloalkenyl,
7) haloalkyl,
8) heteroalkyl,
9) aryl,
10) heteroaryl,
11) heterobicyclyl, or
12) heterocyclyl,
wherein the alkyl, alkenyl, alkynyl and the cycloalkyl and cycloalkenyl are optionally substituted with one or more R¹ substituents; and wherein the aryl, the heteroaryl, the heterocyclyl and the heterobicyclyl are optionally substituted with one or more R² substituents,
or alternatively, R^{A} and R^{B} together with the nitrogen to which each is bonded form a heterocyclic or a heterobicyclic ring;
R¹ is
1) halogen,
2) NO₂,
3) CN,
4) haloalkyl,
5) C₃-C₇ cycloalkyl,
6) aryl,
7) heteroaryl,
8) heterocyclyl,
9) heterobicyclyl,
10) OR⁶,
11) S(O)₂R³,
12) NR⁴R⁵,
13) NR⁴S(O)₂R³,
14) COR⁶,
15) C(O)OR⁶,
16) CONR⁴R⁵,
17) S(O)₂NR⁴R⁵,
18) OC(O)R⁶,
19) SC(O)R³,
20) NR⁶C(O)NR⁴R⁵,
21) heteroalkyl,
22) NR⁶C(NR⁶)NR⁴R⁵, or
23) C(NR⁶)NR⁴R⁵;
wherein the aryl, heteroaryl, heterocyclyl, and heterobicyclyl are optionally substituted with one or more R² substituents;
R² is
1) halogen,
2) NO₂,
3) CN,
4) C₁-C₆ alkyl,
5) C₂-C₆ alkenyl,
6) C₂-C₄ alkynyl,
7) C₃-C₇ cycloalkyl,
8) haloalkyl,
9) OR⁶,
10) NR⁴R⁵,
11) SR⁶,
12) COR⁶,
13) C(O)OR⁶,
14) S(O)₂R³,
15) CONR⁴R⁵,
16) S(O)₂NR⁴R⁵,
17) aryl,
18) heteroaryl,
19) heterocyclyl,
20) heterobicyclyl,
21) heteroalkyl,
22) NR⁶C(NR⁶)NR⁴R⁵, or
23) C(NR⁶)NR⁴R⁵,
wherein the aryl, the heteroaryl, the heterocyclyl, and the heterobicyclyl are optionally substituted with one or more R⁷ substituents;
R³ is
1) C₁-C₆ alkyl,
2) C₂-C₆ alkenyl,
3) C₂-C₄ alkynyl,
4) C₃-C₇ cycloalkyl,
5) haloalkyl,
6) aryl,
7) heteroaryl,
8) heterocyclyl, or
9) heterobicyclyl,
wherein the alkyl, the alkenyl, the alkynyl and the cycloalkyl are optionally substituted with one or more R¹ substituents; and wherein the aryl, the heteroaryl, the heterocyclyl and the heterobicyclyl are optionally substituted with one or more R² substituents;
R⁴ and R⁵ are independently chosen from
1) H,
2) C₁-C₆ alkyl,
3) C₂-C₆ alkenyl,
4) C₂-C₆ alkynyl,
5) aryl,
6) heteroaryl, or
7) heterocyclyl,
or R⁴ and R⁵ together with the nitrogen to which they are bonded form a heterocyclic ring;
R⁶ is
1) H,
2) C₁-C₆ alkyl,
3) C₂-C₆ alkenyl,
4) C₂-C₆ alkynyl,
5) aryl,
6) heteroaryl, or
7) heterocyclyl;
R⁷ is
1) halogen,
2) NO₂,
3) CN,
4) C₁-C₆ alkyl,
5) C₂-C₆ alkenyl,
6) C₂-C₄ alkynyl,
7) C₃-C₇ cycloalkyl,
8) haloalkyl,
9) OR⁶,
10) NR⁴R⁵,
11) SR⁶,
12) COR^{E},
13) C(O)OR⁶,
14) S(O)₂R³,
15) CONR⁴R⁵,
16) S(O)₂NR⁴R⁵,
17) heteroalkyl,
18) NR⁶C(NR⁶)NR⁴R⁵, or
19) C(NR⁶)NR⁴R⁵;
or a salt thereof, or a prodrug thereof;
wherein the following compounds are excluded:
A is H, (Xaa)ₐ is His-D-Trp, R^{A} is H, R^{B} is CH₃, (Xaa′)_{b} is Trp-D-Phe-Lys and B is NH₂;
A is H, (Xaa)ₐ is His-D-Trp-Ala-Trp, R^{A} is H, R^{B} is CH₂Ph, (Xaa′)_{b} is Lys and B is NH₂;
A is H, (Xaa)ₐ is (D/L)-His, R^{A} is H, R^{B} is CH₂-p-C₆H₄OH, (Xaa′)_{b} is Ala-Trp-D-Phe-Lys and B is NH₂;
A is H, (Xaa)ₐ is His-D-Trp-Ala, R^{A} is H, R^{B} is CH₂-p-C₆H₄OH, (Xaa′)_{b} is D-Phe-Lys and B is NH₂; and
A is H, (Xaa)ₐ is His-D-Trp-Ala-D-Phe, R^{A} is H, R^{B} is (CH₂)₄NH_{2, b} is 0, and B is NH₂.

7. A method according to claims 6, wherein a is an integer from 1 to 5 and b is an integer from 1 to 5.

8. The method according to claim 1 wherein said CD36 activator compound is administered at a submicromolar concentration.

9. The method according to claim 8 wherein said CD36 activator compound is administered at a dosage about 300 μg/kg.

10. The method according to claim 1 wherein said CD36 activator compound is administered systemically.

11. The method according to claim 1 wherein said CD36 activator compound is administered intraperitoneally.

12. The method according to claim 1 wherein said CD36 activator compound is administered intravenously.

13. The method according to claim 1 wherein said CD36 activator compound is administered locally.

14. The method according to claim 1 wherein said CD36 activator compound is administered intravitreally.

15. The method according to claim 1 wherein said CD36 activator compound is administered intraocularly.

16-44. (canceled)

45. The method according to claim 1 wherein said CD36 activator compound is a CD36 activator antibody.

46. The method according to claim 1 wherein said CD36 activator compound is a growth hormone-releasing peptide derivative.

47. A method of determining whether a test compound may be useful for the prevention or treatment of dry age-related macular degeneration, said method comprising
(a) contacting said test compound with a cell expressing CD36; and
(b) determining whether CD36 expression and/or activity is increased in the presence of said compound;
wherein an increase in CD36 expression and/or activity is indicative that said test compound may be useful for the prevention or treatment of dry age-related macular degeneration.


SEQUENCE LISTING (8 sequences)

SEQ ID NO: 1 - DNA, 22 bp, artificial sequence
  primer
aaagaaagggtgtaaaacgcag

SEQ ID NO: 2 - DNA, 22 bp, artificial sequence
  primer
aaagacctctatgccaacacag

SEQ ID NO: 3 - DNA, 20 bp, artificial sequence
  primer
gacaatcaaaagggaagttg

SEQ ID NO: 4 - DNA, 20 bp, artificial sequence
  primer
cctctctgtttaaccttgat

SEQ ID NO: 5 - DNA, 18 bp, artificial sequence
  primer
tgggatggtccttgcctc

SEQ ID NO: 6 - DNA, 20 bp, artificial sequence
  primer
tcgctggagtacacggtggt

SEQ ID NO: 7 - DNA, 22 bp, artificial sequence
  primer
tgctaccatctggcttcgggag

SEQ ID NO: 8 - DNA, 20 bp, artificial sequence
  primer
acccctcaggtgttgcacgt